(19) United States
(12) Patent Application Publication
(10) Pub. No.: US 2018/0207113 A1
(43) Pub. Date: Jul. 26, 2018

(54) VIVO INTRACELLULAR REPROGRAMMING COMPOSITION AND METHOD OF MAKING AND USING SAME

(71) Applicant: Vincent C Giampapa, Montclair, NJ (US)

(72) Inventor: Vincent C Giampapa, Montclair, NJ (US)

(21) Appl. No.: 15/416,207

(22) Filed: Jan. 26, 2017

(51) Int. Cl. A61K 31/155; A61K 31/235; A61K 36/74; A61K 36/906; A61K 31/12; A61K 9/00; A61K 47/06; A23L 33/10

(52) U.S. Cl. CPC A61K 31/155; A61K 31/235; A61K 36/74; A61K 36/906; A23V 2002/00; A61K 9/0092; A61K 9/0053; A61K 47/06; A23L 33/10; A61K 31/12

(57) ABSTRACT

An in vivo intracellular reprogramming method for both somatic and stem cell pools in the mammal's body using, C60 fullerenes can be added to enhance mitochondrial ATP production and Biogenesis, Metformin, Everolimus, Seligilene, Danasitab, Venetoclaxalong with Quercitin may act as agent to enhance autophagy and apoptosis helping to decrease their ongoing formation and a novel resveratrol complex along ribonucleasies worth together to help decrease heterchromation dysregulation aid in DNA repair enhancement and nutraceuticals is disclosed. The present in vivo reprogramming invention works at the epigenetic level and promotor region of the genome inside the nucleus of the cell, and other intracellular organelles, rather than outside the body. The key genetic activity changes that the invention reverses have now been found responsible for causing aging in adult stem cells and somatic cells, the combination of the above compounds have been shown to cause improved and corrected epigenetic changes or reprogramming in vivo in gene expression to induce the necessary genetic changes to improve cellular aging and epigenetic IC changes responsible for the changes and therefore effect total body and cellular health from the organ and tissue level on upward. The key to and extended to a long and healthy life is keeping your somatic cells health and maintaining a pool of stem cells can be called on for acute situations like healing form surgery or trauma or an immune boast to frequent infection.

### FIELD OF THE INVENTION

The present disclosure generally relates to compositions and methods for improving the cellular health of a subject. More specifically this disclosure relates to compositions and methods for epigenetic modification of mammalian cells and cellular reprogramming method for both stem cells and somantic cells using medicinals including metformin, everolomus or rapamycin analogs and a novel sirtuin complex and dna repair compounds from the vine uncaria tomentosa or other cats claw extract whether hot water or alcohol extracts nutraceuticals.

### BACKGROUND OF THE INVENTION

The Cellular aging process based on recent research can now be viewed as a dynamic process as a continuum which can be altered in both a positive or negative fashion. We now understand that we inherit genetic tendencies not genetic certainties. Our gene activity can be altered by what is now known as the Science of Epigenetics or the ability to alter gene activity by what we consume and experience through our environment.

### Problem Solved

The present invention improves the ability of mammalian cells to mimic calorie restriction and reduce cellular senescence, maintain cellular function more optimally in both aging mammalian somatic and stem cells, add DNA repair capacity, and enhances mitochondrial ATP production. It also stresses the importance of decreasing the formation of senescent cells as well as removing these old damaged cells to decrease the influence of their by-products on their neighboring healthy cells.

Previous combinations of medical drugs or neutracueticals either alone or in previous combinations have not been able to affect efficiently the genetic origin of decreased cellular function in aging mammalian cells. This unique combination of compounds utilizes the most recent research detailing changes in gene expression that are related to cellular aging and loss of function in aging mammalian cells in both somatic and stem cell pools. This process is accomplished in vivo at the epigenetic level and without the need for cellular removal and external reprogramming with viruses or complicated cellular manipulation or cell cultures.

The present in vivo reprogramming invention works at the epigenetic level inside the nucleus of the cell, and other intracellular organelles, rather than outside the body. A number of epigenetic mechanisms may be involved including but not limited to methylation-or-De-Methelation, Phosphorylation, acetylation of the Histine complexes of DNA or the promoter regions of DNA or both. The key genetic changes that the invention reverses have now been found responsible for causing aging in adult Stem Cells and somatic cells, the combination of the above compounds have been shown to improve and partially cause the correct epigenetic changes or reprogramming in vivo in gene expression to induce the necessary genetic and protein as well as RNA and MicroRNA and Exosomal product formation that can induce the changes to improve cellular aging and therefore effect total body and cellular health.

Aging is an important risk factor for most chronic diseases and is the primary factor for the majority of morbidity and health care expenditures in developed nations. Decreased cellular function associated with cellular senescence results in the disorders and dysfunctions typically associated with aging mammalian cells. A potent inducer of cellular senescence is (epi) genomic stress, which can result from direct DNA damage, dysfunctional telomeres, disrupted chromatin or strong mitogenic signals. Additionally cellular senescence can cause chronic inflammation through the senescence-associated secretory factors (SASF).

There exists an ongoing need for compositions and methods that improve cellular functions that have been negatively impacted due to one or mechanisms associated with cellular senescence. Further there exists an ongoing need for compositions and methods to improve the cellular health of a subject.

In 2014, the PCT generated an International Search Report in which the principal reference cited was U.S. 2012/0177730 (2012) to Baron et al of Elcelyx Therapeutics, Inc., entitled Chemosensory Receptor Ligand-Based Therapies Based. The search person equates the biguanide or substituted biguanide of PCT application with some of the bitter receptor ligand compositions suggested by Baron, particularly with reference to his ¶s [0397], [0435], and in Example 40 at p. 182, ¶ [1739]. “Biguanide” is a very broad term in biochemistry, and seemingly was able to equate a biguanide to a bitter receptor ligand. Ligand is also a broad term but bitter receptor ligand is a narrow subset thereof.

Suggested is a narrower species of biguanide. The broad definition of the PCT application opened the door to this issue, given that various benefits of bitter receptor ligands are well known (see pp. 65-72 of Baron) and that these ligands take many forms. On a more positive note, Baron's claims (see p. 185) looks very different, meaning there apparently is no infringement issue. We re-defined the scope of the biguanides in the epigenic modification system (EMF) such that they do not resemble a bitter receptor ligand, the U.S. Examiner will have to reconsider Baron and conduct a de novo search of the prior art, if we convert to National Phase.

Another issue is that Baron defines a bitter receptor ligand in so many different ways that the term is nearly devoid of meaning. On the other hand one might recite limitations of use of biguanides to commercially attractive options and such that it does not appear that you are attempting to pre-empt the use of metaformin or phenaformin for any purpose. The system is re-defined within the scope of the biguanides in the epigenic modification system (EMF) such that they do not resemble a bitter receptor ligand. Another issue is that Baron defines a bitter receptor ligand in so many different ways that the term is nearly devoid of meaning. On the other hand one might recite limitations of use of biguanides to commercially attractive options and such that it does not appear pre-empted use of metaformin or phenaformin for any purpose.

Baron at U.S. 2014/0030332 Chemosensory Receptor Ligand-Based Therapies. Provided herein are methods for treating conditions associated with a chemosensory receptor, including diabetes, obesity, and other metabolic diseases, disorders or conditions administering a composition comprising a chemosensory receptor ligland, such as a bitter receptor ligland. Also provided herein is chemosensory receptor ligand composition including bitter receptor ligand composition, and methods for preparation thereof for use in the method of the present method of the present invention. Also provided herein are compositions comprising metformin and salts thereof and methods of use.

Baron et al, at US 2014/0341986. Biguanide Compositions and Methods of Treating Metabolic Disorders. Provided herein are methods for treating certain conditions including diabetes, obesity, and other metabolic diseases, disorders or conditions by administrating a composition comprising a biguanide or related heterocyclic composing, e.g., metorormin. Also provided herein are hetercyclic compound compositions, and methods for the preparation thereof for use in the methods of the present invention. Also provided herein are compositions comprising metoformin and salts thereof and methods of use.

Sagman, et al, is U.S. Pat. No. 7,758,889, Fullernes in Targeted Therapies (2010). The present invention relates to a composition comprising a C,-Ab, wherein C is a fullerene or nanotube comprising a carbon atoms and Ab is a moiety comprising an antigen-binding site and is linked to the C. The composition can further comprise a therapeutic molecule associated with the C.

In another embodiment of Sugman, the present invention relates to a method of treating a disease in a mammal, comprising administrating to the mammal an effective amount of composition comprising (i) a C-Ab. Wherein C is a fullerence or nanotube comprising a carbon atoms and Ab is a moiety comprising an antigen-binding site and is linked to the C and (ii) a pharmaceutically acceptable carrier.

Mitophagy is the selective degradation of mitochondria by autophagy. If often occurs to defective mitochondria following damage or stress. For example, the liver may contain mitochondrial fragments. “As part of almost every lysosome in these glucagon-treated cells it is possible to recognize a mitochondrian or a remnant of one. It was also mentioned in 1977 by scientists studying metamorphosis in silkworms, “mitochondria develop functional alterations which would activate autophagy.” Mitophagy is key in keeping the cell healthy. It promotes turnover of mitochondria and prevents accumulation of dysfunctional mitochondria which can lead to cellular degeneration.

Organelles and buts of cytoplasm are sequestered and targeted for degradation by the lysosome for hydrolytic digestion by a process known as autophagy. Mitochondria metabolism leads to the creation of by-products that lead to DNA damage and mutations. Therefore, a healthy population of mitochondria is critical for the well-being of cells. Previously it was thought that targeted degradation of mitochondria was a stochastic event, but accumulating evidence suggests that mitophagy is a selective process.

### SUMMARY OF THE INVENTION

Disclosed herein is a composition comprising a pharmaceutical, a nutraceutical and an optional carbonaceous material wherein the pharmaceutical comprises a biguanide or a substituted biguanide. Further described herein is the method of making and using same.

The combination of unique neutraceuticals C60 fullerenes and the medications Eveolimus, Metformin, Venetoclax, and Inisatab work synergistically producing an enhanced gene profile effect when used together than they would separately due to the multiple genetic pathways triggered simultaneously and the synergistic interaction of these key pathways as a system more similar to a youthful cell genetic profile. This youthful genetic cell profile is not present in aging human cells due to DNA damage and accumulation of intracellular debris due to the aging process in general including free radicals and altered gene function caused by the normal aging process at the cellular level. When any of the compounds are given on separate bases, there is a loss of synergy or multiple systems effected and the results of the multiple genetic profiles that are reprogrammed at the same time, is not present.

The invention can be made by combining the correct combination C60 fullerenes, nutraceuticals, and Metformin, with any number of stand delivery oral incipient agents either in capsule, tablet or liquid form for oral consumption or IV or intra nasal or intrathecal use.

The key necessary elements are Metformin, Everolimus, Resveratrol, and Polyphenol ribonucleosides, sirtuin stacks of reservatrol or its analogs, complexes Fixation, Pterostelbene, and cats claw extract, of the Uncaria Sinesis Halvi or Tomentosa varieties. C60 fullerenes can be added to make the Invention function better due to the effect it has on opening the hilstone proteins more efficiently and that then allowing access to the other compounds to interact as the epigenetic levels at the promotor region of the gene or effect the opening of the histone winding at the key points. The other antioxidant compounds enhance the intrinsic antioxidant gene production SOD Catalase and Glutathione Peroxidase and levels that are not normally present in aging mammalian cells. The correct amount and combination of antioxidants also helps regulate the NRF-2 pathway which controls the intrinsic antioxidant pathways. At the first line of defense, to quench free radical damage from its primary source of the mitochondria.

It is imperative that special forms of Resveratrol from the Japanese Knot Weed and polyphenol complexes like PTERSTILBENE who interacts on a different gene location to work syngestically with Reservatrol be used for the invention to function optimally and this should be combined with the Metform and other RX meds mentioned above as the initial step to initiate the general genetic in vivo reprogramming process within the nucleus and mitochondria of the cell.

Herein “epigenetic” refers to the heritable changes in gene activity and expression that occurs without alternation in DNA sequence. Nonlimiting examples of epigenetic modifications include posttranslational modifications such as DNA methylation and chromatin remodeling and histone modification. Herein “improved cellular function” refers to characteristics such as an elevated level of expression of genes that promote cell viability, enhanced cell viability, and/or a reduced presence of materials detrimental to the functioning of the cell. Such compositions and methods are collectively termed “epigenetic modification systems” and designated EMS.

In an embodiment, an EMS composition comprises at least one pharmaceutical, at least one nutraceutical and optionally a carbonaceous material. Herein a “pharmaceutical” refers to any chemical substance formulated or compounded that is intended for internal, or external use in the medical diagnosis, cure, treatment, or prevention of a disease, disorder, or dysfunction. Herein a “nutraceutical” refers to any substance that is a food or part of a food and provides medical or health benefits including the prevention and treatment of disease.

In an embodiment, the EMS comprises at least one pharmaceutical that functions to mimic caloric restriction in mammals. In an embodiment, the EMS comprises an antihyperglycemic agent, also known as an anti-diabetic agent. In an embodiment, the EMS comprises a biguanide or a substituted biguanide. Biguanides are utilized in the treatment of type 2 diabetes which is also known as non-insulin-dependent diabetes mellitus (NIDDM). Biguanides are known to lower both the hyperglycemia and the hyperinsulinemia associated with NIDDM and may function to enhance insulin-receptor activation and downstream signaling and activate the liver kinase B I-5′AMP-activated protein kinase signaling pathway.

Galega officinalis (French lilac) was used in diabetes treatment for centuries.^{[3]} In the 1920s, guanidine compounds were discovered in Galega extracts. Animal studies showed that these compounds lowered blood glucose levels. Some less toxic derivatives, synthalin A and synthalin B, were used for diabetes treatment, but after the discovery of insulin, their use declined. Biguanides were reintroduced into Type 2 diabetes treatment in the late 1950s. Initially phenformin was widely used, but its potential for sometimes fatal lactic acidosis resulted in its withdrawal from most pharmacopeias (in the U.S. in 1978).^{[4]} Metformin has a much better safety profile, and it is the principal biguanide drug used in pharmacotherapy worldwide.

Biguanides do not affect the output of insulin, unlike other hypoglycemic agents such as sulfonylureas and meglitinides. Therefore, they are effective in Type 2 diabetics; and in Type 1 diabetes when used in conjunction with insulin therapy.

The mechanism of action of biguanides is not fully understood, eg many mechanisms have been proposed for metformin.
Mainly used in Type II Diabetes, metformin is considered to increase insulin sensitivity in vivo, resulting in reduced plasma glucose concentrations, increased glucose uptake, and decreased gluconeogenesis.
However, in hyperinsulinemia, biguanides can lower fasting levels of insulin in plasma. Their therapeutic uses derive from their tendency to reduce gluconeogenesis in the liver, and, as a result, reduce the level of glucose in the blood. Biguanides also tend to make the cells of the body more willing to absorb glucose already present in the blood stream, and there again reducing the level of glucose in the plasma.

The most common side effect is diarrhea and dyspepsia, occurring in up to 30% of patients. The most important and serious side effect is lactic acidosis, therefore metformin is contraindicated in renal insufficiency. Renal functions should be assessed before starting metformin. Phenformin and buformin are more prone to cause acidosis than metformin; therefore they have been practically replaced by it. However, when metformin is combined with other drugs (combination therapy), hypoglycemia and other side effects are possible.

Some biguanides are also used as antimalarial drugs. Examples include:

Proquanil

Chlorproquanil

### Disinfectants

The disinfectants chlorhexidine, polyaminopropyl biganide (PAPB), polihexanide, and alexindine feature biguanide functional groups.

We have identified urolithin A (UA) as a first-in-class natural compound that induces mitophagy both in vitro and in vivo following oral consumption. In C. elegans, UA prevented the accumulation of dysfunctional mitochondria with age and extended lifespan. Likewise, UA prolonged normal activity during aging in C. elegans, including mobility and pharyngeal pumping, while maintaining mitochondrial respiratory capacity.

### DETAILED DESCRIPTION OF THE INVENTION

As stated above, the present invention Improves the ability of mammalian cells to mimic calorie restriction and reduce cellular senescence maintain cellular function including DNA repair, and ATP production and cellular autophagy more optimally in both aging mammalian somatic and stem cells The invention claimed here solves this problem. The need to maintain cellular aging function and correct gene expression patterns is critical in mammals and humans due to the fact that they are born with a fixed number of stem cells to replace damaged and worn somatic or body cells. By maintaining stem cell pools in more optimal health and function the somatic cells can reform their jobs of maintaining organ and tissue function longer and maintain a state of general body health and quality of life longer.

The Invention solves this problem by a combination of oral compounds using both a combination of drugs and unique group of nutraceuticals and new form of carbon molecules called C60 fullerenes that effect different genetic pathways now known to be involved in cellular aging. The synergistic effect of the correct combination of both medial drugs and natural compounds called Nutraceuticals are what creates or mimics more closely a more youthful or younger gene profile in the mammal treated with the regimen described within mammalian cells to more efficiently mimic, function, and repair the accumulated damage with the cell itself, calorie restriction and reduce cellular senescence. This is accomplished by this combination of compounds by changing the gene expression of the pathways controlled by but not limited to the following genes: MTOR, AMPK, DNA repair genes, Keep/nrf2, P53, P13k, and P16 INK. The list of the key genes the invention helps to regulate are the following MTOR, IGF-1, AMPK, DNA repair genes, P16, P21, P53, NFK-B, TNF-A, PUMA-BAX, TGF-B, among others Thru the unique combination of the effects of these neutraceuticals, Carbon compound and medical drugs create in effect a more efficient in vivo gene reprogramming process than is normally present in aged or older and or damaged stem cells and body cells.

The claimed invention differs from what currently exists. This approach is different than existing cellular aging methods in that it combines a number of drugs with a long history of safety in human metformin-use with the latest categories of nutraceuticals and with C60 fullerenes that have been shown to be effective in improving cellular aging and function. The invention is a new form of in in vivo cellular reprogramming. Other methods require ex vivo processes that are more expensive and have high potential complications. As well as create potential complications to the host through possible infection and or contamination of the cells while in the extracorporeal state prior to reentry to the original donor or into the patient being treated.

This invention is an improvement on what currently exists. This approach is different than existing cellular antiaging methods in that it combines a drug with a long history of safety in human metaformin-use with the latest neutraceuticals and with C60 fullerenes that have been shown to be effective in improving cellular aging and function in many animal models. The invention is a new form of In-vivo cellular reprogramming. Other methods require ex vivo processes that are more expensive and have high potential complications.

The past attempts at improving cellular aging did not affect cells efficiently at the genetic or epigenetic level in vivo but utilize external cellular processes and then return the cells back to the subject for therapeutic effects. The present Invention utilizes in vivo reprogramming which does not require cells to be removed or replaced or physically altered outside the body and works at the epigenetic level which is the origin of the proteins that are formed from our genetic sequences and that are do for the epigenetic effects that initiate the changes at the cellular level to alter gene expression and mimic calorie restriction, reduce cell senescence, limit malignant cell transformation, and improve cellular DNA repair. It is also most likely that the reprogrammed cells positively impact and improve the “stem cellniche” and will also help in restoring stem cell function in this manner as well.

The present in vivo reprogramming invention works at the epigenetic level inside the nucleus of the cell, and other intracellular organelles, rather than outside the body. The key genetic changes that the invention reverses have now been found responsible for causing aging in adult stem cells and somatic cells jointly as well as producing the pro inflammatory compounds that effect healthy neighboring cells and that cause them to age more rapidly. The combination of the above compounds have been shown to cause the correct specific epigenetic changes or reprogramming in vivo In gene expression to induce the necessary genetic changes to improve cellular aging and therefore effect total body and cellular health. One of the key purposes of the invention is to remove senescent or aged cells from the patient as well as decrease the formation of new senescent cells by turning down the gene expression of the gene MTOR. This gene has been studied as is known to increase senescent cell formation as a mammal ages or has accumulated cell damage due to chemotherapy or radiation therapy.

Also, this invention can be used to treat diabetes, mental acuity decline, weight gain, memory loss and vision loss which changes are also partially due to senescent cell accumulation. It can be used to enhance cell culture of aging mammalian cells as well as anticaking and antiglycation therapy in general to enhance both stem cell and somatic cell function

The Version of the Invention Discussed Includes:
1. Metformin as the base compound that mimics calorie restriction gene down regulates glucose levels and IGF-1 levels as well as augments AMPK level profiles in order to positively reprogram specific genes. Everolimus or analogs of rapamycin that inhibit MTOR and therefore slow the production of senescent cell formation as well as help remove these cells through augmentation of apoptosis and autophagy DASATINIB, NAVITICLAX—also called ABT-136 and Quercitien all help with removing senescent cells via a synergistic approach by inducing apoptosis.
2. Resveratrol and polyphenol complexes have been shown to interact with situtin genes that are involved in DNA repair and that may help the SIRT genes return to their promotor regins more efficiently therefore avoiding heterochromation dysregulation and histone malfunction and therefore accelerated aging in the cell. In the form of red grape complex, trans-resveratrol in any form or related compounds, and ptereostilbene to initiate the genetic changes that inhibit cell senescence and support calorie restriction profiles quercetin that HLPS increase autophagy.
3. Cats claw extract has unique qualities that enhance DNA repair both single and double strand repair, inhibit inflammation compounds including NFK-B and TNF-A as well as other OPOR inflammatory cytokines as well as, augment apoptosis, and the removal of senescence cells.
4. Curcumin, ginger, compounds induce intracellular genes to produce intrinsic antioxidants—the cells first line of defense against free radical production in the mitochondria. This is augmented with the use of Xeligeline or Deprynel.
5. C60 FULLERENES position themselves intracellularly due to their content of low density olive oil and the bucky ball configuration of the fullerene which acts as a sink to capture free radicals at the source of their product as well in the nucleus of the cell and the mitochondria to enhance gene expression Involved In energy production and intracellular antioxidant production which includes the formation of SOD.CATALASE, and Glutathione Peroxidase. This allows the cell to function at a much higher efficiency so the general genetic processes a produce more proteins and the correct variety of protein products like RNA, mRNA, microvessicels, and exosomes essential for cell function and repair. The use of Sirtuin analogs that enhance ATP production in the mitochondria and the use of ribonucleaside compounds to further enhance DNA repair and ATP production have been shown in recent animal studies to be very effective. In augmenting cellular energy, they enhance ATP production.

Relationship Between the Components:

Each component initiates changes in the cellular maintenance and repair process in a synergistic and systems MYLTI synergistic fashion that can do more with individual compounds used approach format.

Compound-1 Metformin causes gene expression changes in the nucleus of the cell is a broad general profile mimicking calorie restriction changes in general. As well as decreasing the IGF-1 pathway and keeping insulin levels more stable and lower. It also augments mitochondrial biogenesis-increasing the number of mitochondria within the cells as they age.

Compound 2—Resveratrol and Polyphenol complexes target the specific genes involved in cell senescence and DNA repair like sirtuin family of genes and also work synergistically with Metformin to enhance Calorie Restriction Profiles.

Compound 3—Cats Claw extract in the specific form of AC-11 or CMED-100 has been show to augment the DNA Repair process both double and single strand breaks and double strand breaks being the most difficult to repair and pertaining to UV and UA radiation levels as well as aid removal of senescence cells by turning on the genes Involved in Apoptosis or programmed cell death. It also inhibits inflammatory compounds—NFKAPPA-BETA and TNF-ALPHA which are involved in and are triggering events in telomere shortening. A study to measure b-galactocidase levels in a wbc population per hpf would help document this.

Compound 4—antinflammatory and antioxidant compounds like ginger and curcumin and turmeric stimulate the genes Involved in intrinsic antioxidant production outside the nucleus and n the mitochondria to increase the cells level of intrinsic antioxidants SOD, Catalase glutathione peroxidase—the cells first line of defense against DNA Damage and gene inhibition. Quercitin also actively encourages autophagy or removal of toxic cell debris inside the cell. Seligene-called deprynl orally at 5 m per 5 days per week has been shown to augment the same intridic antioxidants as well.

Compound 5—C60 fullerenes found in extra virgin olive oil bases have shown remarkable improvements in mitochondrial ATP production and have recently been shown to penetrate all aspects of Human cells including the nucleus where the work by improving epigenetic changes thru alteration of the histones that control the exposure of genes to key compounds that then either turn on or turn off gene activity. Fullerenes also help enhance ATP production In the cell so it functions more efficiently without producing more free radicals. In summary the chain of events described above create a genetic profile that is more like a youthful functioning cell with similar genetic profile and without the damaged gene expression patterns found in old or diseased cells. These changes that are effected have been shown to be present in both somatic and stem cells.

In an embodiment, the biguanide comprises 2-(N-phenethylcarbamimidoyl)guanidine (phenformin) characterized by Formula 1 or a pharmaceutically acceptable salt thereof, N,N-dimethylimidodicarbonimidic diamide (metformin) characterized by Formula 2 or a pharmaceutically acceptable salt thereof, or combinations thereof. In an embodiment, the EMS comprises metformin or a pharmaceutically acceptable salt thereof, or alternatively metformin hydrochloride.

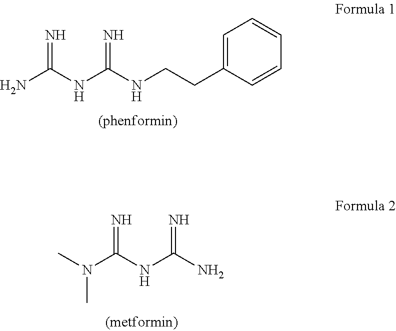

The pharmaceutical (e.g., metformin) may be present in the EMS in an amount of from about 0.1 weight percent (wt. %) to about 99 wt. % based on the total weight of the EMS, alternatively from about 5 wt. % to about 50 wt. %, or alternatively about 0.1, 0.25, 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 99 wt. %.

In an embodiment, the EMS comprises at least one nutraceutical. The nutraceutical may comprise a polyphenol or polyphenol complex. Herein polyphenols, also known as polyhydroxyphenols, refer to a structural class of compounds characterized by the presence of more than one phenol unit (−). In an embodiment the polyphenols or polyphenol complexes are derived from natural sources, alternatively the polyphenols or polyphenol complexes are synthetically produced. In an embodiment the polyphenol comprises a stilbenoid or a hydroxylated derivative of stilbene. For example the EMS may comprise reservatrol, piceatannol, pinosylvin, ptereostilbene, or combinations thereof. The structural formulas of a stilbene, reservatrol, picetannol, pinosylvin, and ptereostilbene are presented as Formulas 3, 4, 5, 6, and 7 respectively.

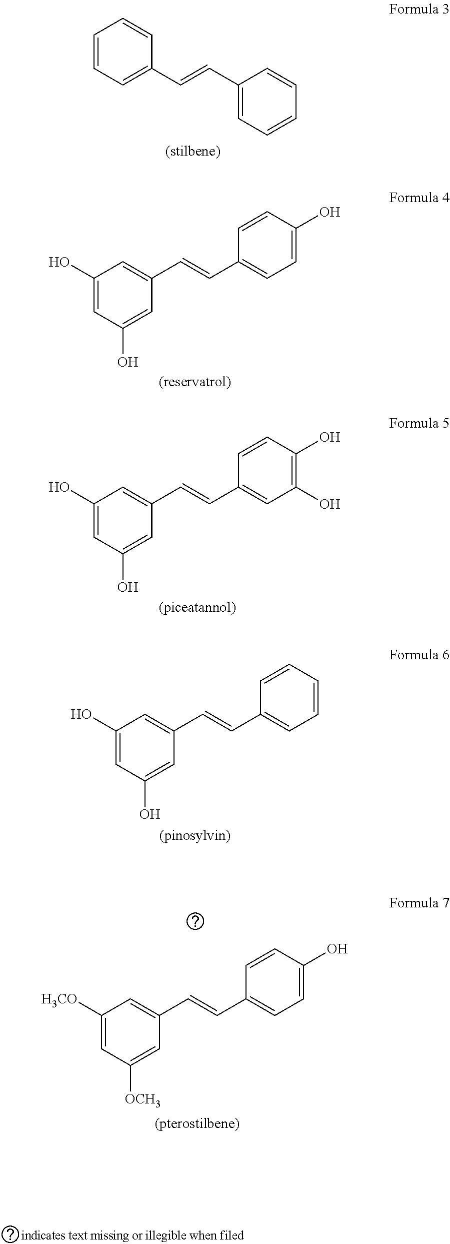

In an embodiment, the nutraceutical comprises an extract of Uncaria tormentosa or a related plant species. Uncaria tormentosa is a woody vine native to the Peruvian Amazon Basin. Extracts of this plant exhibit biological activity such as antiinflammatory activity, immunomodulation and anti-oxidant activity. Without wishing to be limited by theory the biological activity of extracts of Uncaria tormentosa or related species may be attributable in part to the presence of various oxindole alkaloids, triterpenes, and quinovic acid glycosides. Thus in an embodiment, the nutraceutical comprises oxindole alkaloids, triterpenes, quinovic acid glycosides, or combinations thereof structurally similar or structurally equivalent to those found in extracts of Uncaria tormentosa.

Alternatively, the nutraceutical comprises extracts of Uncaria tormentosa or a related plant species. Any suitable extract of Uncaria tormentosa or a related plant species may be employed in the nutraceutical. For example, the Uncaria tormentosa extract may be a hydroalcoholic extract (e.g., a hydroethanolic extract), a freeze-dried extract, or an aqueous extract. In an embodiment, an aqueous extract of Uncaria tormentosa that is substantially free of oxindole alkaloids is employed in the neutraceutrical. In an embodiment an aqueous extract of Uncaria tormentosa suitable for use in the present disclosure has oxindole alkaloids present in an amount of less than about 10 weight percent (wt. %) based on the total weight of the extract, alternatively less about 9, 8, 7, 6, 5, 4, 3, 2, 1, 0.5, 0.1, or 0.05 wt. %.

In an embodiment the nutraceutical comprises compounds that function to induce one or more intracellular genes to produce intrinsic antioxidants. For example the nutraceutical comprises a compound or material derived or obtained from the plant family Zingiber aceae. In an embodiment the nutraceutical comprises curcumin. Curcumin is a diarylheptanoid that is the principal component of the s ice turmeric. The enol form of curcumin is depicted in Formula 8.

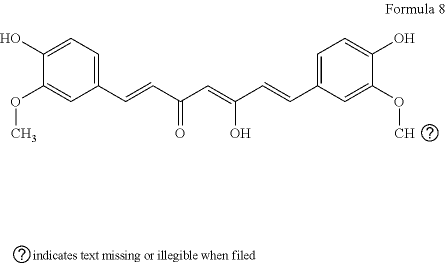 [SMILES: COc1cc(/C=C/C(=O)/C=C(O)/C=C/c2ccc(O)c(OC)c2)ccc1O]

In an embodiment the nutraceutical comprises ginger. Ginger or ginger root is the thizome of the plant Zingiber officinale. [0017] In an embodiment the nutraceutical comprises a telomere maintenance complex comprising a group of components that individually and/or jointly support maintaining telomeres of the chromosome. The telomere maintenance complex may include, but is not limited to, purslane extract (aerial parts), turmeric rhizome extract containing curcuminoids, quercetin dehydrate, cayenne pepper fruit, vanadium, fenugreek seed, astragalus root extract, valerian root, omega fatty acid complex including linoleic acid, alpha-linolenic acid, and oleic acid, borage seed oil, evening primrose oil, fish body oil or combinations thereof.

In an embodiment the nutraceutical comprises a calorie restriction mimetic and gene expression complex comprising a group of components that individually and/or jointly support calorie restriction mimetics and gene expression. The calorie restriction mimetics and gene expression complex may include, but is not limited to, trans-resveratrol, such as from extract of polygonum cuspidatum root or grape skin, pterostilbene, fisetin, alpha lipoic acid, coenzyme Q-10, betaine HCl, sulfur, such as from methylsulfonylmethane, L-carnitine tartrate, L-carnitine HCl, acetyl-L-carnitine, avocado extract, or combinations thereof.

In an embodiment the nutraceutical comprises free radical scavenger complex comprising a group of components that individually and/or jointly function as an antioxidant and reduces free radicals. The free radical scavenger complex may include, but is not limited to, green tea leaf extract containing catechin and polyphenols, anthocyanins such as from bilberry fruit and grape skin extracts, ginkgo biloba leaf extract containing ginkgo flavonglycosides and sesquiterpene lactones, cruciferous vegetable concentrate (broccoli, kale, radish) containing glucosinolates, grape skin extract containing total phenolics, tomato lycopene extract containing lycopene, rosemary extract (aerial parts), pycnogenol (pine bark extract), lutein such as from marigold flower extract, green barley grass (aerial parts), or combinations thereof.

In an embodiment, the nutraceutical comprises a DNA repair complex comprising a group of components that individually and/or jointly support DNA repair in both nuclei and mitochondria. The DNA repair complex may include, but is not limited to, water extract of Uncaria tomentosa containing carboxy alkyl esters, N-acetyl-cysteine, inositol hexaphosphate, melatonin, or combinations thereof.

In an embodiment, the nutraceutical comprises a stem cell maintenance complex comprising a group of components that individually and/or jointly support optimal maintenance of stem cells and gene expression. The stem cell maintenance complex may include, but is not limited to, chlorella algae, spirulina algae, klamath blue-green algae, fuxoxanthin seaweed (whole plant), nori seaweed extract or brown seaweed extract, or combinations thereof.

In an embodiment the nutraceutical comprises a cell regulation complex comprising a group of components that individually and/or jointly support optimal cell regulation. The cell regulation complex may include, but is not limited to, gotu kola leaf, phosphatidyl choline such as from soy lecithin, DMAE bitartrate, cordyceps sinensis mushroom extract containing cordyceptic acid, royal jelly 3× containing 10-HDA, 1-glutamine, taurine, L-phenylalanine, L-tyrosine, inositol, L-arginine, L-ornithine, guarana seed extract, probiotic blend (lactobacillus acidophilus, lactobacillus plantarum, bifidobacterium bifidum and lactobacillus casei), amylase, neutral protease, cellulase, lactase, lipase, or combinations thereof.

In an embodiment the nutraceutical comprises a vitamin. Examples of vitamins suitable for use in the nutraceutical include, but are not limited to, vitamin A, vitamin C, vitamin D, vitamin E, vitamin K, thiamin, riboflavin, niacin, vitamin B6, folate, vitamin B12, biotin, pantothenic acid, or combinations thereof.

In an embodiment the nutraceutical comprises minerals including, but not limited to, iodine, calcium, zinc, selenium, copper, manganese, chromium, molybdenum, or combinations thereof. Optionally, the minerals may further include transitional metal trace elements such as iridium, rhodium, ruthenium, or combinations thereof.

In an embodiment the nutraceutical is present in the EMS in an amount from about 0.1 wt. % to about 99 wt. % based on the total weight of the EMS, alternatively from about 5 wt. % to about 50 wt. %, or alternatively about 0.1, 0.25, 0.5, 1, 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 99 wt. %.

In an embodiment, the EMS optionally comprises a carbonaceous material, alternatively a fullerene. Fullerenes comprise any carbonaceous material having a structure of a regular, three-dimensional network of fused carbon rings. Such a network of fused carbon rings can be arranged in any suitable structure. Nonlimiting examples of such structures include cylindrical, spherical, ovoid, oblate and oblong structures. Typical fullerenes include cylindrical carbon nanotubes and icosahedral C60 carbon molecules. In an embodiment the fullerene comprises a C60 of the type depicted in Formula 9.

Fullerene molecules are acceptably non-toxic and are known to be rendered less hydrophobic by the addition of moieties such hydroxyl or carboxylic acid groups. In an embodiment, a fullerene suitable for use in the EMS has been functionalized to increase the hydrophillicity of the molecule. In an embodiment, the EMS comprises a hydroxylated fullerene, a carboxylated fullerene or both.

In an embodiment the fullerene is present in the EMS in an amount of from about 0.1 wt. % to about 10 wt. % based on the total weight of the EMS, alternatively from about 5 wt. % to about 10 wt. %, or alternatively from about 0.1, 0.25, 0.5, 1, 2, 3, 4, 5, 6, 7, 8, 9, or 10 wt. %

In an embodiment, this disclosure provides for pharmaceutical compositions comprising an EMS of the type described herein and a pharmaceutically acceptable carrier. The term “pharmaceutical composition”, is intended to encompass a product comprising the active ingredient(s) (e.g., an EMS of the type described herein), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing an EMS of the type described herein, additional active ingredient(s), and pharmaceutically acceptable excipients.

In an embodiment the EMS comprises suitable processing and/or manufacturing additives that facilitate preparation of suitable dosing forms of the compositions disclosed herein. For example, the EMS may comprise binders. Binders are substances used to cause adhesion of particles in granulations. In an embodiment the binder functions to facilitate the preparation of a suitable form of the EMS. Binders suitable for use in the present disclosure include by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol or combinations thereof.

In an embodiment the EMS comprises a diluent. Diluents suitable for use in the present disclosure include, by way of example and without limitation, microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin, or combinations thereof.

In an embodiment the EMS comprises a lubricant. Lubricants are substances used in composition formulations that reduce friction during production of a suitable form of the composition. Lubricants suitable for use in the present disclosure include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, or combinations thereof.

In an embodiment the EMS optionally comprises a colorant. As used herein, the term “colorant” refers to compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and combinations thereof. Coloring agents also can include pigments, dyes, tints, titanium dioxide, natural coloring agents, such as grape skin extract, beet red powder, beta carotene, annato, carmine, turmeric, paprika or combinations thereof.

In an embodiment, the EMS comprises a sweetener. Examples of sweeteners suitable for use in the present disclosure include without limitation dextrose, erythritol, fructose, glycerin, glucose, inositol, isomalt, lactitol, lactose, maltitol, maltose, mannitol, sorbitol, sucrose, tagatose, trehalose, xylitol, and combinations thereof.

Binders, diluents, lubricants, colorants, sweeteners, and other processing additives may be included singularly or in combination in any suitable amount effective to meet one or more user and/or process needs.

Any suitable route of administration may be employed for providing an organism (e.g., human or animal) with an effective dosage of an EMS of the type described herein. For example, oral, sublingual, rectal, parenteral, pulmonary, and the like may be employed. Dosage forms include tablets, troches, dispersions, suspensions, solutions, capsules and the like. The most suitable route in any given case will depend on the nature and severity of the conditions being treated and on the nature of the active ingredient. A pharmaceutical composition comprising an EMS may be conveniently presented in unit dosage form and prepared by any suitable methodology. Alternatively the EMS is prepared in forms for intravenous, intranasal or intrathecal delivery.

An EMS of the type described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions (comprising the EMS) for oral dosage form, any of suitable pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets. Because of their ease of administration, tablets and capsules represent the most advantageous oral dosage unit form in which case solid pharmaceutical carriers may be employed. If desired, tablets comprising the EMS may be coated by standard aqueous or nonaqueous techniques.

In an embodiment pharmaceutical compositions comprising an EMS of the type described herein suitable for oral administration are presented as discrete units such as capsules, cachets or tablets each containing a predetermined amount of the active ingredient (e.g., EMS), as a powder or granules or as a solution or a suspension in an aqueous liquid, a non-aqueous liquid, an oil-in-water emulsion or a water-in-oil liquid emulsion. Such compositions may be prepared by any suitable methodology but all methods include the step of bringing into association the active ingredient with the carrier. In general, the compositions are prepared by uniformly and intimately admixing the active ingredient (e.g., EMS) with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient (e.g., EMS) in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

In an embodiment, the EMS is prepared in a solid dosage form, such as a tablet, a capsule, a sachet, etc., or any therapeutically acceptable form. In an embodiment, an EMS of the type disclosed herein prepared in a solid oral dosage form (e.g., capsule) may be sugar-coated or enteric-coated by any suitable methodology. The unit dosage forms may be individually wrapped, packaged as multiple units on paper strips or in vials of any size, without limitation. In an embodiment, an EMS of the type disclosed herein is provided to a subject as multiple oral dosage formulations wherein a first formulation comprises the nutraceutical and optional carbonaceous material (e.g., fullerene) and a second formulation comprises the pharmaceutical agent. In an embodiment the EMS is be packaged in unit dose, rolls, bulk bottles, blister packs and combinations thereof, without limitation.

In an embodiment, a subject (e.g., human) may be administered or may self-administer any of the EMS compositions described herein for some duration of time. For example the subject may self-administer the EMS from about 1 to about 5 times daily, alternatively one time daily, alternatively two times daily, alternatively three times daily, alternatively four times daily, or alternatively five times daily. In an embodiment, the EMS is administered or self-administered at a dose suitable to provide an effective amount or therapeutically effective amount of the EMS for a period of time. The terms “effective amount” or “therapeutically effective amount,” as used herein, refer to a sufficient amount of the EMS which will relieve to some extent one or more of the symptoms of the disease or condition being treated or will provide the outcomes described (i.e., quantitative or qualitative improvements in a subject's cellular health). An appropriate “effective” amount in any individual case may be determined using techniques, such as a dose escalation study. An EMS of the type described herein may be used in combination with other drugs that are used in the treatment/prevention/suppression or amelioration of the adverse health events for which an EMS of the type described herein are useful.

In an embodiment, the EMS is administered to provide a dose that result in qualitative and/or quantitative improvement in a subject's health and/or cellular function for a period of days, weeks, months or years. In an embodiment, a subject may receive a standard dose, (e.g., a dosage formulation manufactured and commercially available that provides some preweighed amount of the EMS composition) from 1-5 times daily for a time period of 24 hours to 5 years, alternatively from 7 days to 5 years, alternatively from 30 days to 5 years, alternatively from 60 days to 5 years, alternatively from 90 days to 5 years, alternatively from 120 days to 5 years, alternatively from 180 days to 5 years, alternatively from 210 days to 5 years, alternatively from 240 days to 5 years, alternatively from 365 days to 5 years, alternatively from 60 days to 365 days, or alternatively from 30 days to 120 days. In an embodiment, an EMS formulation (as a single oral dose formulation, or as the sum of multiple oral dose formulations) may be provided in an amount of from about 10 mg to about 5000 mg, alternatively from about 250 mg to about 5000 mg, alternatively from about 500 mg to about 5000 mg, alternatively from about 750 mg to about 5000 mg, alternatively from about 1000 mg to about 1500 mg, alternatively from about 250 mg to about 1500 mg, alternatively from about 500 mg to about 1500 mg, alternatively from about 750 mg to about 1500 mg, or alternatively from about 250 mg to about 1500 mg.

In an embodiment the EMS provides a unique combination of nutraceuticals, pharmaceuticals and carbonaceous material. In an embodiment, the pharmaceutical comprises metformin, the nutraceutical comprises a fullerene is optionally present in the composition. Metformin works to synergistically produce an enhanced gene profile effect and when used together than they would separately due to the multiple genetic pathways triggered simultaneously and the interaction of these key pathways as a system more similar to a youthful cell genetic profile. This youthful genetic cell profile is not present in aging human cells due to DNA damage. Free radicals and altered gene function caused by the normal aging process at the cellular level. When any of the compounds are given on separate bases, there is a loss of synergy or the system effects and the results of the multiple genetic profiles that are reprogrammed at the same time, is not present.

In an embodiment the EMS, provided in an oral dosage formulation, is used as an in vivo cellular reprogramming therapy taken from once to four times daily. Herein the term “reprogramming” refers to a process by which one or more functions of a cell are altered from a first state, a, to one or more later states (a′, a″, b, etc. . . . ) displaying improved cellular function.

In an aspect the bioavailability of the EMS is such that that EMS may be administered with or without food. It can also be used intravenously or intranasally or intrathecally via the standard medical routes normally utilized for therapy of this sort.

In an embodiment, the EMS can be used in whole or in part as a cell culture media or as an ex vivo reprogramming method for aging mammalian cells in the laboratory setting, either for research or therapeutic purposes. For example, the EMS may be contacted with a population of cells having a high ratio of senescent cells to nonsenescent cells. In such embodiments, upon contact with the EMS, at least a portion of the senescent cells may display increased cell viability and functionality, reduced expression of senescence markers, increased mitochondrial function, reduced expression of inflammatory genes, increased expression of DNA repair genes, or combinations thereof. The extent of increased cell viability and functionality, reduced expression of senescence markers, increased mitochondrial function, reduced expression of inflammatory genes, increased expression of DNA repair genes may be determined using any suitable methodology and compared to an otherwise similar cell population not contacted with an EMS of the type disclosed herein.

In an embodiment, the methodologies disclosed herein are therapeutic and involve a subject experiencing an undesirable condition. In an embodiment the methodologies disclosed herein result in therapies that are prophylactic, palliative, curative, or combinations thereof. Methodologies and compositions of the type disclosed herein may be utilized in the treatment of a wide variety of undesirable conditions related to decreases in cellular function and viability such as neurological disorders; autoimmune diseases; and disorders associated with radiation overexposure (chronic or acute). In an embodiment, the EMS may function as a therapeutic in the treatment of disorders such as diabetes, mental acuity decline, weight gain, memory loss and vision loss. It can be used to enhance cell culture of aging mammalian cells as well as anti-aging therapy in general and to enhance both stem cell and somatic cell function.

It is contemplated the methodologies and compositions disclosed herein may result in an increased expression of genes associated with beneficial cellular events with a concomitant decrease in the expression of genes associated with adverse cellular events. In some embodiments, the methodologies and compositions disclosed herein result in an increased expression of genes associated with beneficial cellular events.

Urolithins extend lifespan and improve fitness in C. elegans. Treatment with EA at the same concentration had no effect on lifespan this, it was decided to continue with a deeper investigation of urolithins, and focused on UA, as it is the most prevalent EA-derived metabolite observed in humans. A clear dose-response effect was found on lifespan when UA concentrations were increased from 10 to 50 μM. At 50 μM, UA significantly delayed the mortality observed at advanced ages.

The following are enumerated embodiments which are provided as non-limiting examples:

A first embodiment which is a composition comprising a pharmaceutical, a nutraceutical and an optional carbonaceous material wherein the pharmaceutical comprises a biguanide or a substituted biguanide.

A second embodiment which is the composition of the first embodiment wherein the biguanide or substituted biguanide comprise a compound represented by the following structure:

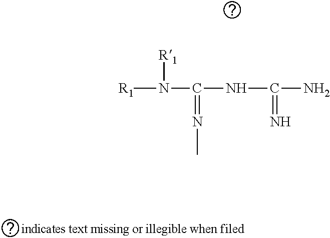

where R_{1?} Rr, and R₂ are each independently hydrogen, a hydrocarbyl group, or an organyl group. [0056] A third embodiment which is the composition of any of the first through second embodiments wherein the pharmaceutical comprises 2-(N-phenethylcarbamimidoyl)guanidine, N,N-dimethylimidodicarbonimidic diamide, pharmaceutically acceptable salts thereof, or combinations thereof.

A fourth embodiment which is the composition of any of the first through third embodiments wherein the pharmaceutical is present in an amount of from about 0.1 wt. % to about 99 wt. %.

A fifth embodiment which is the composition of any of the first through fourth embodiments wherein the nutraceutical comprises a polyphenol, a polyphenol complex, or combinations thereof.

A sixth embodiment which is the composition of any of the first through fifth embodiments wherein the nutraceutical comprises stilbene, reservatrol, piceatannol, pinosylvin, ptereostilbene, or combinations thereof.

A seventh embodiment which is the composition of any of the first through sixth embodiments wherein the nutraceutical comprises an extract of Uncaria tormentosa.

An eighth embodiment which is the composition of the seventh embodiment wherein the extract is an aqueous extract.

A ninth embodiment which is the composition of the eighth embodiment wherein the aqueous extract comprise less than about 10 wt. % of oxindole alkaloids based on the total weight of the extract.

A tenth embodiment which is the composition of any of the first through ninth embodiments wherein the nutraceutical comprises ginger, curcumin, or combinations thereof.

An eleventh embodiment which is the composition of any of the first through tenth embodiments wherein the nutraceutical comprises a telomere maintenance complex, a calorie restriction mimetic and gene expression complex, a free radical scavenger complex, a DNA repair complex, a stem cell maintenance complex, a cell regulation complex, a vitamin, a mineral, or combinations thereof.

A twelfth embodiment which is the composition of any of the first through eleventh embodiments wherein the nutraceutical is present in the EMS in an amount of from about 0.1 wt. % to about 99 wt. %.

A thirteenth embodiment which is the composition of any of the first through twelfth embodiments wherein the carbonaceous materials comprises a fullerene.

A fourteenth embodiment which is the composition of the thirteenth embodiment wherein the fullerene is functionalized.

A fifteenth embodiment which is the composition of any of the thirteenth through fourteenth embodiments wherein the fullerene is present in an amount of from about 0.1 wt. % to about 10 wt. % based on the total weight of the composition.

A sixteenth embodiment which is the composition of any of the first through fifteenth embodiments further comprising a binder, a diluent, a lubricant, a colorant, a sweetener, or combinations thereof.

A seventeenth embodiment which is an oral formulation of the composition any of the first through sixteenth embodiments.

An eighteenth embodiment which is a method comprising administering to a subject the oral formulation of the seventeenth embodiment about one times to about five times daily.

A nineteenth embodiment which is the method of the eighteenth embodiment wherein the oral formulation is administered for a period of from about 24 hours to about 5 years.

A twentieth embodiment which is the method of any of the eighteenth through nineteenth embodiments wherein the subject is experiencing a disorder selected from the group consisting of diabetes, obesity, mental acuity decline, memory loss, vision loss, or combinations thereof.

A twenty-first embodiment which is a composition comprising metformin, a telomere maintenance complex, a calorie restriction mimetic and gene expression complex, a free radical scavenger complex, a DNA repair complex, a stem cell maintenance complex, a cell regulation complex, a vitamin, a mineral, and optionally a fullerene.

While embodiments of the disclosure have been shown and described, modifications thereof can be made without departing from the spirit and teachings of the disclosure. The embodiments described herein are exemplary only, and are not intended to be limiting. Many variations and modifications of the disclosure disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Nl, and an upper limit, Nu, is disclosed, any number falling within the range is specifically disclosed. In particular, the following numbers within the range are specifically disclosed: N=N1+k*(Nu−Nl), wherein k is a variable ranging from 1 percent to 100 percent with a 1 percent increment, i.e., k is 1 percent, 2 percent, 3 percent, 4 percent, 5 percent, 50 percent, 51 percent, 52 percent, 95 percent, 96 percent, 97 percent, 98 percent, 99 percent, or 100 percent. Moreover, any numerical range defined by two N numbers as defined in the above is also specifically disclosed. Use of the term “optionally” with respect to any element of a claim is intended to mean that the subject element is required, or alternatively, is not required. Both alternatives are intended to be within the scope of the claim.

Use of broader terms such as comprises, includes, having, etc. should be understood to provide support for narrower terms such as consisting of, consisting essentially of, comprised substantially of, etc. Particularly, the transitional term “comprising”, which is synonymous with “including,” “containing,” “having,” or “characterized by,” is inclusive or open-ended and does not exclude additional, unrecited elements or method steps. The transitional phrase “consisting of excludes any element, step, or ingredient not specified in the claim. The transitional phrase “consisting essentially of limits the scope of a claim to the specified materials or steps and those that do not materially affect the basic and novel characteristic(s) of the claimed invention. A “consisting essentially of claim occupies a middle ground between closed claims that are written in a “consisting of format and fully open claims that are drafted in a “comprising” format. Absent an indication to the contrary, when describing a compound or composition “consisting essentially of is not to be construed as “comprising,” but is intended to describe the recited component that includes materials which do not significantly alter the composition or method to which the term is applied. While compositions and methods are described in terms of “comprising” various components or steps, the compositions and methods can also “consist essentially of or “consist of the various components or steps.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the embodiments of the present disclosure. The discussion of a reference herein is not an admission that it is prior art to the present disclosure, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A composition comprising a pharmaceutical, a nutraceutical and an optional carbonaceous material wherein the pharmaceutical comprises a biguanide or a substituted biguanide.

2. The composition of claim 1 wherein the biguanide or substituted biguanide comprise a compound represented by the following structure: 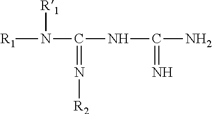
where R_{1?} Rr, and R₂ are each independently hydrogen, a hydrocarbyl group, or an organyl group.

3. The composition of claim 1 wherein the pharmaceutical comprises 2-(N-phenethylcarbamimidoyl)guanidine, N,N-dimethylimidodicarbonimidic diamide, pharmaceutically acceptable salts thereof, or combinations thereof.

4. The composition of claim 1 wherein the nutraceutical comprises a polyphenol, a polyphenol complex, or combinations thereof.

5. The composition of claim 1 wherein the nutraceutical comprises stilbene, reservatrol, piceatannol, pinosylvin, ptereostilbene, or combinations thereof.

6. The composition of claim 1 wherein the nutraceutical comprises an extract of Uncaria tormentosa.

7. The composition of claim 6 wherein the extract is an aqueous extract.

8. The composition of claim 7 wherein the aqueous extract comprise less than about 10 wt. % of oxindole alkaloids based on the total weight of the extract.

9. The composition of claim 1 wherein the nutraceutical comprises ginger,
curcumin, or combinations thereof.

10. The composition of claim 1 wherein the nutraceutical comprises a telomeraintenance complex, a calorie restriction mimetic and gene expression complex, a free radical scavenger complex, a DNA repair complex, a stem cell maintenance complex, a cell regulation complex, a vitamin, a mineral, or combinations thereof.

11. The composition of claim 1 wherein the carbonaceous materials comprises a fullerene.

12. The composition of claim 11 wherein the fullerene is functionalized.

13. The composition of claim 11 wherein the fullerene is present in an amount of from about 0.1 wt. % to about 10 wt. % based on the total weight of the composition.

14. The composition of claim 1 further comprising a binder, a diluent, a lubricant, a colorant, a sweetener, or combinations thereof.

15. An oral formulation of claim 1.

16. A method comprising administering to a subject the oral formulation of claim 15 from about one times to about five times daily.

17. The method of claim 18 wherein the subject is experiencing a disorder selected from the group consisting of diabetes, obesity, mental acuity decline, memory loss, vision loss, or combinations thereof.

18. The composition of claim 1 wherein the nutraceutical comprises a telomeraintenance complex, gene expression complex, a free radical scavenger complex, a DNA repair complex, a stem cell maintenance complex, a urolithin mitochondria, or combinations thereof.

19. A composition comprising metformin, a telomere maintenance complex, a calorie restriction mimetic, gene expression complex, a free radical scavenger complex, a DNA repair complex, a stem cell maintenance complex, a urolithin mitochondria, and optionally a fullerene.
